(19) United States
(12) Patent Application Publication
(10) Pub. No.: US 2025/0329013 A1
(43) Pub. Date: Oct. 23, 2025

(54) METHOD AND DEVICE FOR AUTOMATED BRAIN WHITE MATTER FIBER TRACT SEGMENTATION COMBINED WITH ANATOMICAL PRIORS

(71) Applicant: Beijing University of Posts and Telecommunications, Beijing (CN)

(72) Inventors: Fangrong ZONG, Beijing (CN); Di ZHANG, Beijing (CN)

(21) Appl. No.: 19/180,559

(22) Filed: Apr. 16, 2025

(30) Foreign Application Priority Data
Apr. 17, 2024 (CN) 202410464500.4

(51) Int. Cl. G06T 7/00; G06T 7/11; G06T 7/143; G06V 10/26; G06V 10/46; G06V 10/764; G06V 10/776; G06V 20/70; G16H 30/20

(52) U.S. Cl. CPC G06T 7/0012; G06T 7/11; G06T 7/143; G06V 10/273; G06V 10/46; G06V 10/764; G06V 10/776; G06V 20/70; G16H 30/20; G06T 2207/10092; G06T 2207/20021; G06T 2207/20076; G06T 2207/20081; G06T 2207/30016; G06V 2201/031

(57) ABSTRACT

Provided are a method and device for automated brain white matter fiber tract segmentation combined with anatomical priors. The method includes: obtaining whole-brain fiber point coordinates and structural T1-weighted magnetic resonance images, determining superficial white matter fibers and deep white matter fibers based on the whole-brain fiber point coordinates, and generating an anatomical brain region division map based on the structural T1-weighted magnetic resonance images; determining an individual-level anatomical feature descriptor of each fiber based on the superficial white matter fibers, the deep white matter fibers and the anatomical brain region division map, and respectively determining a cluster-level anatomical feature descriptor corresponding to each fiber; and inputting the whole-brain fiber point coordinates, the individual-level anatomical feature descriptors and the cluster-level anatomical feature descriptors into a trained fiber tract segmentation model, and obtaining classification results of fiber tracts.

This application claims priority of Chinese Patent Application No. CN202410464500.4, filed on Apr. 17, 2024, which is hereby incorporated by reference in its entirety.

### TECHNICAL FIELD

The present disclosure relates to the technical field of magnetic resonance imaging and, in particular, to a method and device for automated brain white matter fiber tract segmentation combined with anatomical priors.

### BACKGROUND

According to the technique of diffusion magnetic resonance imaging, a non-uniform linearly distributed magnetic field is generated by a pulsed magnetic field gradient. This results in sensitive changes in the signal intensity in the gradient direction when water molecules diffuse, thereby the water molecule diffusion coefficient in the gradient direction can be measured to obtain a set of diffusion-weighted image data. Therefore, the diffusion magnetic resonance imaging can depict the structure of living tissue orientation and reconstruct fibers.

Fiber tract segmentation refers to segmenting whole-brain fibers into different clusters according to the geometric shape, anatomical features or functional features of the fibers. Fiber tract segmentation is the basis of brain science research. The more accurate the segmentation, the more useful it is for downstream research tasks. With the development of artificial intelligence technology, neural network segmentation models have demonstrated higher efficiency in segmenting fiber tracts.

Currently, there are two conventional fiber tract segmentation methods: voxel-based semantic segmentation and fiber streamline-based segmentation. The voxel-based semantic segmentation requires voxel-level masks of fiber tracts, and the annotation of the voxel-level masks is complex and time-consuming. The fiber streamline-based segmentation treats fibers as streamlines formed by sequences of connected points, and the annotation of each streamline can be obtained by clustering and other methods. Moreover, the annotation of streamlines contains shape information such as the coordinates of the points and the curvature of the streamline, which is not reachable for the voxel-based semantic segmentation. In fiber streamline-based segmentation methods, there are generally two steps: obtaining feature descriptors of fibers and using the feature descriptors to build corresponding segmentation methods. The feature descriptors of fibers often come from geometric information and are, for example, built on the basis of point coordinates, curvature and the like.

Although the conventional fiber tract segmentation methods mentioned above can achieve efficient segmentation of fiber tracts, the feature descriptors of fibers only take into account the geometric features of the fiber streamlines, but not the features of the fibers in anatomical brain regions, resulting in low accuracy in the segmentation results of the fiber tracts. Therefore, how to improve the accuracy of the classification results of whole-brain white matter fiber tracts is a technical problem that needs to be solved urgently.

### SUMMARY

In view of the foregoing, embodiments of the present disclosure provide a method and device for automated brain white matter fiber tract segmentation combined with anatomical priors to eliminate or ameliorate one or more of the defects present in the prior art.

An aspect of the present disclosure provides a method for automated brain white matter fiber tract segmentation combined with anatomical priors. The method includes the following steps:
obtaining whole-brain fiber point coordinates and structural T1-weighted magnetic resonance images, determining superficial white matter fibers and deep white matter fibers based on the whole-brain fiber point coordinates, and generating an anatomical brain region division map based on the structural T1-weighted magnetic resonance images;
determining an individual-level anatomical feature descriptor of each fiber based on the superficial white matter fibers, the deep white matter fibers and the anatomical brain region division map, and respectively determining a cluster-level anatomical feature descriptor corresponding to each fiber based on the individual-level anatomical feature descriptor of each of the superficial white matter fibers and the individual-level anatomical feature descriptor of each of the deep white matter fibers; and
inputting the whole-brain fiber point coordinates, the individual-level anatomical feature descriptors and the cluster-level anatomical feature descriptors into a trained fiber tract segmentation model, and obtaining classification results of fiber tracts based on the trained fiber tract segmentation model.

In some embodiments of the present disclosure, the determining an individual-level anatomical feature descriptor of each fiber based on the superficial white matter fibers, the deep white matter fibers and the anatomical brain region division map includes:
performing 3D affine transformation on each of the superficial white matter fibers and the deep white matter fibers, and mapping fiber points of each fiber to brain regions of the anatomical brain region division map to obtain an anatomical distribution of the fiber points of each fiber; and
obtaining an individual-level anatomical feature descriptor of each fiber based on the anatomical distribution of fiber points of each fiber.

In some embodiments of the present disclosure, the respectively determining a cluster-level anatomical feature descriptor corresponding to each fiber based on the individual-level anatomical feature descriptor of each of the superficial white matter fibers and the individual-level anatomical feature descriptor of each of the deep white matter fibers includes:
performing hierarchical clustering on the superficial white matter fibers and the deep white matter fibers respectively based on the individual-level anatomical feature descriptor of each of the superficial white matter fibers and the individual-level anatomical feature descriptor of each of the deep white matter fibers;
determining the number of clusters corresponding to the superficial white matter fibers and the number of clusters corresponding to the deep white matter fibers; and
determining clusters to which various fibers belong based on the number of clusters corresponding to the superficial white matter fibers, the number of clusters corresponding to the deep white matter fibers and hierarchical clustering results, and determining a cluster-level anatomical feature descriptor corresponding to each fiber based on the cluster to which each fiber belongs.

In some embodiments of the present disclosure, the fiber tract segmentation model includes a point cloud encoder module, a first embedding layer, a second embedding layer and a decoder module; and/or,
the anatomical brain region division map contains 286 brain regions.

In some embodiments of the present disclosure, the inputting the whole-brain fiber point coordinates, the individual-level anatomical feature descriptors and the cluster-level anatomical feature descriptors into a trained fiber tract segmentation model and obtaining classification results of fiber tracts based on the trained fiber tract segmentation model includes:
performing local feature encoding and global feature encoding on the whole-brain fiber point coordinates on the basis of the point cloud encoder, and fusing the local feature encoding and global feature encoding to obtain feature codes;
inputting the individual-level anatomical feature descriptors into the first embedding layer to obtain first embedding codes;
inputting the cluster-level anatomical feature descriptors into the second embedding layer to obtain second embedding codes;
fusing the feature codes, the first embedding codes and the second embedding codes to obtain fused codes; and
inputting the fused codes to the decoder to obtain classification results of fiber tracts.

In some embodiments of the present disclosure, the method further includes:
building an initial fiber tract segmentation model;
building a loss function and a sample data set for the model; and
pre-training the initial fiber tract segmentation model based on the loss function and the sample data set of the model to obtain a trained fiber tract segmentation model.

In some embodiments of the present disclosure, the loss function is:

$$Loss = \frac{1}{N} {\sum }_{i} {Loss}_{i} = - \frac{1}{N} {\sum }_{i} {\sum }_{c = 1}^{M} {y}_{i c} log ( {p}_{i c} ) ;$$

where, M represents the number of categories into which the fiber tracts are segmented; y_{ic} is 0 or 1, y_{ic} is 1 when a predicted category result of fiber i is the same as a real category result, and y_{ic} is 0 when the predicted category result of fiber i is different from the real category result; N represents a total number of fibers, and pic represents a probability that fiber i belongs to fiber tract category c.

In some embodiments of the present disclosure, the determining superficial white matter fibers and deep white matter fibers based on the whole-brain fiber point coordinates includes:
inputting the whole-brain fiber point coordinates into a trained fiber classification model for pre-classification to obtain a superficial white matter fiber pre-classification result and a deep white matter fiber pre-classification result;
inputting superficial white matter fibers in the superficial white matter fiber pre-classification result into a fiber filtration model to obtain false superficial white matter fibers; and
labeling the false superficial white matter fibers as deep white matter fibers.

According to another aspect of the present disclosure, further disclosed is a system for automated brain white matter fiber tract segmentation combined with anatomical priors, including: a processor, a memory and a computer program stored on the memory. The processor is configured to execute the computer program, and the system implements the steps of the method as described in any of the above embodiments when the computer program is executed.

According to yet another aspect of the present disclosure, also disclosed is a computer-readable storage medium having stored thereon a computer program. When the computer program is executed by a processor, the steps of the method as described in any of the above embodiments are implemented.

According to the method and device for automated brain white matter fiber tract segmentation combined with anatomical priors described herein, an anatomical brain region division map is generated based on individual structural T1-weighted magnetic resonance images, and feature descriptors with anatomical information of fibers are built. That is, based on the obtained white matter fiber annotation data and the corresponding individual anatomical brain region division map, the anatomical feature descriptors of fibers in a sample are calculated and built to achieve accurate segmentation of white matter fiber tracts. Compared with the conventional method that performs fiber segmentation only based on the geometric features of fibers, the anatomy-guided fiber tract segmentation method of the present disclosure has more detailed constraints and more accurate segmentation results, providing a foundation for subsequent brain science research.

Additional advantages, objectives, and features of the present disclosure will be set forth in part in the following description, and will in part become apparent to those skilled in the art after studying the following or may be learned from practice of the present disclosure. The objectives and other advantages of the present disclosure can be achieved and obtained by the structures specifically indicated in the specification and the drawings.

Those skilled in the art will understand that the objectives and advantages that can be achieved with the present disclosure are not limited to those specifically described above, and the above and other objectives that can be achieved with the present disclosure will be more clearly understood based on the following detailed description.

### BRIEF DESCRIPTION OF DRAWINGS

The drawings described herein are used to provide further understanding of the present disclosure, constitute a part of the present application, and do not constitute a limitation on the present disclosure. In the drawings:

FIG. 1 is a schematic flow chart of a method for automated brain white matter fiber tract segmentation combined with anatomical priors according to an embodiment of the present disclosure.

FIG. 2 is a schematic flow chart of a method for automated brain white matter fiber tract segmentation combined with anatomical priors according to another embodiment of the present disclosure.

FIG. 3 is a schematic architecture diagram of a system for automated brain white matter fiber tract segmentation combined with anatomical priors according to an embodiment of the present disclosure.

### DESCRIPTION OF EMBODIMENTS

In order to make the objectives, technical solutions and advantages of the present disclosure more clear, the present disclosure is further described in detail below in conjunction with embodiments and the accompanying drawings. Here, the schematic embodiments and the description of the present disclosure are for explaining the present disclosure, but not intended to limit the present disclosure.

It should also be noted that, in order to avoid obscuring the present disclosure due to unnecessary details, only the structures and/or processing steps closely related to the solution according to the present disclosure are shown in the drawings, while other details that are not closely related to the present disclosure are omitted.

It should be emphasized that the term “include/comprise”, when used herein, refers to the presence of features, elements, steps or components, but does not exclude the presence or addition of one or more other features, elements, steps or components.

It should also be noted that, if not specifically stated, the term “connect” used herein refers not only to direct connection, but also to indirect connection with the presence of an intermediate.

According to the conventional white matter fiber tract segmentation methods, fiber annotations are obtained by using a fiber tracking algorithm to obtain a whole-brain fiber map of each sample and then performing clustering to generate multiple white matter fiber tracts. Each fiber in each sample has its own annotation. Although the conventional white matter fiber tract segmentation methods can complete the segmentation of fiber tracts, the obtained fiber tracts do not conform to anatomical knowledge and are still far from fine-grained and accurate segmentation. During research, the inventors of the present disclosure found that the conventional white matter fiber tract segmentation methods cannot achieve fine-grained and accurate segmentation. The main reasons are as follows: First, there always are millions of whole-brain fiber streamlines of a single sample obtained by fiber tracking. Each streamline contains a large number of sampling points. With a huge amount of data, segmentation relying on geometric information such as the position and shape of fiber streamlines is a major challenge faced by conventional machine learning methods. Second, the introduction of anatomical knowledge is a difficult process. Each fiber has its own brain regions where it starts, passes through and ends, and for a same brain region there are multiple fibers pass through. How to give anatomical feature descriptions to the whole-brain fibers is an unresolved issue at present.

In order to realize fine-grained and accurate segmentation of fiber tracts, the present disclosure discloses a method and device for automated brain white matter fiber tract segmentation combined with anatomical priors. In the method, the individual anatomical brain region division map for structural T1-weighted magnetic resonance images of each sample is obtained according to the anatomical brain atlas. Based on the individual fiber annotation data and the anatomical brain region division map, new feature descriptors and new neural network models are built.

As used herein, the terms “fiber tracking”, “anatomical brain atlas”, “superficial white matter” and “deep white matter” are explained as follows:
fiber tracking: a process of reconstructing white matter fibers of the brain from diffusion magnetic resonance images. Based on the characteristics of diffusion magnetic resonance imaging, this process can detect the degree and direction of anisotropy of water molecules diffusing in tissue in vivo, estimate the distribution of fiber orientation in each voxel in the image, and further select the most likely orientation according to the probability of connection between a selected seed voxel and other voxels, thereby obtaining the whole-brain fiber connection. There are two types of fiber tracking: deterministic fiber tracking and probabilistic fiber tracking. The deterministic fiber tracking assumes there is a unique fiber direction estimate in each voxel, and provides a single path starting from each seed voxel. The probabilistic fiber tracking takes into account imaging noise, artifacts and model errors, and generates a large set of possible trajectories from seed voxels, with the trajectories containing higher densities being considered to have higher probability of connection.

Anatomical brain atlas: a brain region division map used for analyzing structural T1-weighted magnetic resonance images. Brain regions are often divided according to structure, function, etc. Different brain atlases have different division rules. Among the commonly used brain atlases, the Desikan-Killiany atlas contains 68 cortical regions, with the anatomical locations of the sulci and gyrus on the macro scale being retained. As a fine-grained cortical anatomical region atlas, the Brainnetome atlas contains 246 cortical regions, providing detailed anatomical patterns at the subregional level. The atlas given by Fischl et al. and containing 44 regions is commonly used as a subcortical region atlas. The JHU atlas or ICBM atlas is commonly used as the white matter region atlas.

Superficial white matter and deep white matter: the white matter of the brain is divided into two parts (i.e., superficial white matter and deep white matter) according to whether it is close to the cortex. The superficial white matter is close to the cortex and contains a large number of short-term U-shaped fibers for connecting adjacent or nearby gyrus. The superficial white matter is the white matter area being last myelinated, where oligodendrocytes are more sensitive to metabolic damage. In addition, the superficial white matter contains the highest density of interstitial cells in the white matter, which may develop neurofibrillar tangles. The deep white matter is the area of white matter far away from the cortex, and the fibers therein are divided into three types: connecting fibers, commissural fibers, and projection fibers. Connecting fibers connect different anatomical areas of the ipsilateral hemisphere. Commissural fibers connect the cortex of the left and right hemispheres, mainly constituting the corpus callosum. Projection fibers connect the cerebral cortex and subcortical structures, and most of the fibers project radially through the internal capsule to different functional areas of the cerebral cortex.

Embodiments of the present disclosure will be described below with reference to the drawings. In the drawings, the same reference numerals represent the same or similar parts, or the same or similar steps.

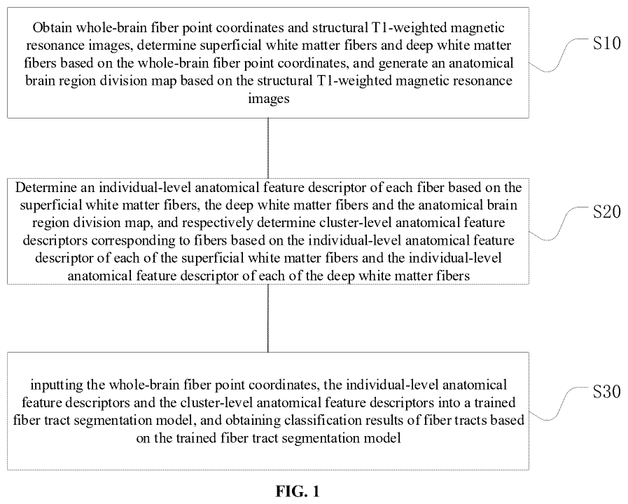

FIG. 1 is a schematic flow chart of the method for automated brain white matter fiber tract segmentation combined with anatomical priors according to an embodiment of the present disclosure. As shown in FIG. 1, the method for automated brain white matter fiber tract segmentation includes at least steps S10 to S30.

Step S10: obtaining whole-brain fiber point coordinates and structural T1-weighted magnetic resonance images, determining superficial white matter fibers and deep white matter fibers based on the whole-brain fiber point coordinates, and generating an anatomical brain region division map based on the structural T1-weighted magnetic resonance images.

In this step, firstly, the whole-brain fiber point coordinates and the structural T1-weighted magnetic resonance images of a subject are obtained; then, based on the obtained whole-brain fiber point coordinates, the fibers are classified to obtain superficial white matter fibers and deep white matter fibers of the subject; and an anatomical brain region division map of the subject is generated based on the obtained structural T1-weighted magnetic resonance images of the subject.

In some embodiments, determining superficial white matter fibers and deep white matter fibers based on the whole-brain fiber point coordinates includes: inputting the whole-brain fiber point coordinates into a trained fiber classification model for pre-classification to obtain a superficial white matter fiber pre-classification result and a deep white matter fiber pre-classification result; inputting superficial white matter fibers from the superficial white matter fiber pre-classification result into a fiber filtration model to obtain false superficial white matter fibers; and labeling the false superficial white matter fibers as deep white matter fibers.

In this embodiment, the classification of superficial white matter fibers and deep white matter fibers of the subject is completed on the basis of the trained fiber classification model and the trained fiber filtration model. During the pre-training process of the fiber classification model and the fiber filtration model, a sample data set needs to be built. An exemplary sample data set includes 100 pieces of sample data, and every piece of sample data includes fiber data and corresponding annotation data. The 100 pieces of sample data may be divided into 5 groups, each containing 20 pieces of sample data. Specifically, every piece of sample data includes fiber point coordinates, fiber labels, label names and structural T1-weighted magnetic resonance images.

During the classification of fibers based on the fiber classification model, superficial white matter fibers and deep white matter fibers are firstly classified in a first stage; and in a second stage, abnormal fibers in the superficial white matter fibers obtained in the first stage are filtered out and classified into deep white matter fibers; and finally superficial white matter fiber data containing 198 labels and deep white matter fiber data containing 602 labels are obtained.

Serving as a neural network used in the first stage to classify superficial white matter fibers and deep white matter fibers, the fiber classification model includes an encoder and an decoder, each composed of a multi-layer perceptron. The input of the network is the whole-brain fiber coordinate points, and the output of the network is the category to which each fiber belongs (e.g., 0 represents superficial white matter and 1 represents deep white matter). Serving as a neural network used in the second stage to filter out false positive superficial white matter fibers, the fiber filtration model is similar to the neural network in the first stage, but the input of the neural network in the second stage is the fiber coordinate points corresponding to the superficial white matter fibers obtained in the first stage, and the output of the neural network in the second stage is the category to which each fiber belongs (e.g., 0 represents a real superficial white matter fiber and 1 represents a false positive superficial white matter fiber, i.e., the superficial white matter fiber that needs to be filtered out).

The structural T1-weighted magnetic resonance images in 100 pieces of sample data may be processed at first to obtain a mean T1-weighted image, and then regions division of the cerebral cortex is obtained using the Brainnetome brain Atlas, and subcortical regions division is obtained using the atlas given by Fischl et al. to anatomize the divided brain regions, thus obtaining an anatomical segmentation map containing a total of 290 brain regions. Since the brain atlas also contains non-gray matter anatomical regions such as left white matter regions, right white matter regions and white matter hypersignal regions, the corresponding label is set to 0 during programming, and a brain region division map containing 286 brain regions is finally obtained and is saved as gm_parc. nii.gz. This brain region division map provides fine-grained anatomical brain regions division and can provide important anatomical guidance for fiber tract segmentation and can greatly improve the accuracy of fiber tract segmentation.

In addition, when this step is implemented based on a computer, an environment may be configured first. For example, to be specific, Python3 programming language and a GPU accelerated PyTorch library may be used. First, an ubuntu18.04 system is downloaded and installed, CUDA and cuDNN related to GPU acceleration are installed, Anaconda3 is installed, a virtual environment is created, and the gpu version of PyTorch and other necessary third-party libraries (such as numpy and antspy) are installed in the virtual environment. In addition, Mrtrix3 and Freesurfer need to be installed for the generation of the anatomical brain region division map.

Prior to calculating the anatomical feature descriptors of fibers, step S10, as a data pre-processing step, obtains the whole-brain fibers with a suitable number of fibers and annotations thereof, as well as the anatomical brain region division map of each sample, and lays a foundation for calculating the anatomical feature descriptors.

Step S20: determining an individual-level anatomical feature descriptor of each fiber based on the superficial white matter fibers, the deep white matter fibers and the anatomical brain region division map, and respectively determining a cluster-level anatomical feature descriptor corresponding to each fiber based on the individual-level anatomical feature descriptor of each of the superficial white matter fibers and the individual-level anatomical feature descriptor of each of the deep white matter fibers.

In this step, anatomical feature descriptors are obtained based on the superficial white matter fibers, deep white matter fibers and anatomical brain region division map obtained in step S10. The anatomical feature descriptors include individual-level anatomical feature descriptors and cluster-level anatomical feature descriptors, realizing digital representation of the relationship between anatomical brain regions and fibers. Specifically, the anatomical feature descriptors of superficial white matter fibers are obtained based on the superficial white matter fibers and the anatomical brain region division map, and the anatomical feature descriptors of deep white matter fibers are obtained based on the deep white matter fibers and the anatomical brain region division map.

As an example, the determining an individual-level anatomical feature descriptor of each fiber based on the superficial white matter fibers, the deep white matter fibers and the anatomical brain region division map includes: performing 3D affine transformation on each of the superficial white matter fibers and the deep white matter fibers, and mapping fiber points of each fiber to the divided brain regions of the anatomical brain region division map to obtain an anatomical distribution of the fiber points of each fiber; and obtaining an individual-level anatomical feature descriptor of each fiber based on the anatomical distribution of fiber points of each fiber.

The respectively determining a cluster-level anatomical feature descriptor corresponding to each fiber based on the individual-level anatomical feature descriptor of each of the superficial white matter fibers and the individual-level anatomical feature descriptor of each of the deep white matter fibers includes: performing hierarchical clustering on the superficial white matter fibers and the deep white matter fibers respectively based on the individual-level anatomical feature descriptor of each of the superficial white matter fibers and the individual-level anatomical feature descriptor of each of the deep white matter fibers; determining the number of clusters corresponding to the superficial white matter fibers and the number of clusters corresponding to the deep white matter fibers; and determining the clusters to which various fibers belongs based on the number of clusters corresponding to the superficial white matter fibers, the number of clusters corresponding to the deep white matter fibers and hierarchical clustering results, and determining a cluster-level anatomical feature descriptor corresponding to each fiber based on the cluster to which each fiber belongs.

In the following embodiments, taking the process of obtaining the anatomical feature descriptors of the superficial white matter fibers as an example, this step is described in detail:

During the process of obtaining individual-level anatomical feature descriptors, firstly, 3D affine transformation is performed on the whole-brain fibers, and the fiber streamlines are mapped onto the anatomical brain region division map. In this embodiment, each fiber has 15 points, each point is mapped to a specific brain region (also known as anatomical brain region), and each brain region has a different number of points. Therefore, the anatomical distribution of points of each fiber is obtained and denoted as individual-level anatomical feature descriptor I.

During the process of cluster-level anatomical feature descriptors, hierarchical clustering is performed on the obtained individual-level anatomical feature descriptors I, and the hierarchical clustering results may be used as rough fiber tracts clustered according to anatomical information, so that the following step S30 can obtain a fine-grained classification result of white matter fiber tracts based on the rough classification of fiber tracts obtained in this step.

As an example, in order to complete the pre-training of the model, 100 pieces of sample data are determined in step S10. In this case, the individual-level anatomical feature descriptors of the superficial white matter fibers corresponding to each piece of sample data may be determined respectively at first, and then the cluster-level anatomical feature descriptors are determined based on the individual-level anatomical feature descriptors corresponding to the 100 pieces of sample data. Specifically, since the number of brain regions in the anatomical brain region division map sample is 286 and the number of points of each fiber is 15, the individual-level anatomical feature descriptor I is a sparse matrix. In this case, the individual-level anatomical feature descriptors I of each sample are added to obtain the overall anatomical feature descriptor S for all the 100 pieces of sample data. As an example, S=

$${\sum }_{n = 1}^{100} {I}_{n} ,$$

Iₙ represents the individual-level anatomical feature descriptor corresponding to the nth sample data. The Euclidean distance between rows in the overall anatomical feature

$${d}_{i j} = \sqrt{{\sum }_{k = 1}^{105} {( {S}_{i , k} - {S}_{j , k} )}^{2}} ,$$

descriptor S is further obtained by: where S_{i,k} represents data corresponding to the kth brain region in row i in the overall anatomical feature descriptor S, S_{j,k} represents data corresponding to the kth brain region in row j in S, and dᵢⱼ represents a distance between row i and row j in S. Two initial clusters Cᵢ and Cⱼ may be obtained by this equation. A distance between Cᵢ and Cⱼ is further calculated for the next step of clustering. In the process of hierarchical clustering, an unweighted pair-group method with arithmetic means (UPGMA) for hierarchical clustering is adopted to recursively merge a new cluster according to the distance between rows in the two clusters. The distance between Cᵢ and Cⱼ is expressed as

$${D}_{{C}_{i} ⋃ {C}_{j , k}} = \frac{\vert {C}_{i} \vert {D}_{i , k} + \vert {C}_{j} \vert {D}_{j , k}}{\vert {C}_{i} \vert + \vert {C}_{j} \vert} ,$$

where |Cᵢ| and |Cⱼ| represent the number of rows in Cᵢ and the number of rows in Cⱼ respectively, and Dᵢₖ and Dⱼₖ represent the distance between row k in Cᵢ and row k in Cⱼ respectively. The above steps are repeated until each of the fibers is affiliated to a cluster to generate a clustering hierarchy tree. After the clustering hierarchy tree is generated, a threshold of the required number of clusters required is specified for segmentation to generate the number of clusters. In the described step of obtaining the anatomical feature descriptors of the superficial white matter fibers, the threshold is set to 8, the superficial white matter fibers are so segmented into 8 clusters, which roughly correspond to the fiber tracts in the regions of frontal lobe, frontal-parietal lobe, parietal lobe, parietal-occipital lobe, parietal-temporal lobe, occipital lobe, occipital-temporal lobe and temporal lobe. In this case, the cluster to which each fiber belongs is labeled to obtain the cluster-level anatomical feature descriptor G of each fiber.

Further, a method similar to the method for obtaining the anatomical feature descriptors of the superficial white matter fibers is used to obtain the anatomical feature descriptors of the deep white matter fibers. After the clustering hierarchy tree corresponding to the deep white matter fibers is obtained, the number of clusters corresponding to the deep white matter fibers is determined. As an example, the deep white matter fibers may be segmented into 34 clusters.

As an example, in the rough classification result of white matter fiber tracts concerned in an embodiment, the superficial white matter fibers are segmented into 8 clusters, and the deep white matter fibers are segmented into 34 clusters. The specific clusters are shown in the following table:

| | | | present in |
|---|---|---|---|
| | | | left and |
| Chinese | | | right |
| full | | | cerebral |
| name | English full name | Abbreviation | hemispheres |
| [symbol] | arcuate fasciculus | AF | ✓ |
| [symbol] | cingulum bundle | CB | ✓ |
| [symbol] | extreme capsule | EmC | ✓ |
| [symbol] | inferior longitudinal fasciculus | ILF | ✓ |
| [symbol] | inferior occipito-frontal | IoFF | ✓ |
| | fasciculus | | |
| [symbol] | middle longitudinal fasciculus | MdLF | ✓ |
| [symbol] | superior longitudinal | SLF I | ✓ |
| | fasciculus I | | |
| [symbol] | superior longitudinal | SLF II | ✓ |
| | fasciculus II | | |
| [symbol] | superior longitudinal | SLF III | ✓ |
| | fasciculus III | | |
| [symbol] | uncinate fasciculus | UF | ✓ |
| [symbol] | cortico-ponto-cerebellar | CPC | ✓ |
| [symbol] | | | |
| [symbol] | inferior cerebellar peduncle | ICP | ✓ |
| [symbol] | intracerebellar input and | Intra-CBLM-I&P | ✓ |
| [symbol] | Purkinje tract | | |
| [symbol] | intracerebellar parallel tract | Intra-CBLM-PaT | ✓ |
| [symbol] | middle cerebellar peduncle | MCP | |
| [symbol] | corpus callosum rostrum | CC 1 | |
| [symbol] | corpus callosum genu | CC 2 | |
| [symbol] | corpus callosum rostral body | CC 3 | |
| [symbol] | corpus callosum anterior | CC 4 | |
| | midbody | | |
| [symbol] | corpus callosum posterior | CC 5 | |
| | midbody | | |
| [symbol] | corpus callosum isthmus | CC 6 | |
| [symbol] | corpus callosum splenium | CC 7 | |
| [symbol] | corticospinal tract | CST | ✓ |
| [symbol] | corona-radiata-frontal | CR-F | ✓ |
| [symbol] | corona-radiata-parietal | CR-P | ✓ |
| [symbol] | striato-frontal | SF | ✓ |
| [symbol] | striato-occipital | SO | ✓ |
| [symbol] | striato-parietal | SP | ✓ |
| [symbol] | thalamo-frontal | TF | ✓ |
| [symbol] | thalamo-occipital | TO | ✓ |
| [symbol] | thalamo-temporal | TT | ✓ |
| [symbol] | thalamo-parietal | TP | ✓ |
| [symbol] | posterior limb of internal | PLIC | ✓ |
| | capsule | | |
| [symbol] | external capsule | EC | ✓ |
| [symbol] | superficial-frontal | Sup-F | ✓ |
| [symbol] | superficial-frontal-parietal | Sup-FP | ✓ |
| [symbol] | superficial-occipital | Sup-O | ✓ |
| [symbol] | superficial-occipital-temporal | Sup-OT | ✓ |
| [symbol] | superficial-parietal | Sup-P | ✓ |
| [symbol] | superficial-parietal-occipital | Sup-PO | ✓ |
| [symbol] | superficial-parietal-temporal | Sup-PT | ✓ |
| [symbol] | superficial-temporal | Sup-T | ✓ |

Step S30: inputting the whole-brain fiber point coordinates, the individual-level anatomical feature descriptors and the cluster-level anatomical feature descriptors into a trained fiber tract segmentation model, and obtaining classification results of fiber tracts based on the trained fiber tract segmentation model.

After the individual-level anatomical feature descriptors and the cluster-level anatomical feature descriptors corresponding to the fibers are obtained based on step S20 described above, the whole-brain fiber point coordinates, the individual-level anatomical feature descriptors and the cluster-level anatomical feature descriptors are further input into the trained fiber tract segmentation model to obtain the fine-grained classification results of the fiber tracts.

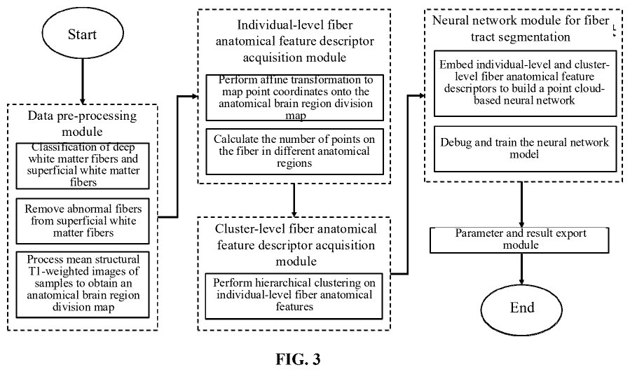

As shown in FIG. 3, the fiber tract segmentation model includes a point cloud encoder module, a first embedding layer, a second embedding layer and a decoder module. That is, the fiber tract segmentation model first performs local feature encoding and global feature encoding on the whole-brain fiber point coordinates on the basis of the point cloud encoder, and fuses the local feature encoding and global feature encoding to obtain feature codes; inputs the individual-level anatomical feature descriptors into the first embedding layer to obtain first embedding codes; inputs the cluster-level anatomical feature descriptors into the second embedding layer to obtain second embedding codes; fuses the feature codes, the first embedding codes and the second embedding codes to obtain fused codes; and inputs the fused codes to the decoder to obtain classification results of fiber tracts. In this embodiment, geometric information on “point coordinates” of fibers is inputted into the point cloud encoder module which is based on a point cloud neural network model and then feature codes are outputted (i.e., point cloud features), the anatomical feature descriptors are also combined with point cloud features through the embedding layers, and finally a fine-grained segmentation result is obtained by the decoder. The superficial white matter fibers are finally segmented into 198 categories, and the deep white matter fibers were finally segmented into 602 categories, realizing the segmentation from rough to fine-grained.

In the above embodiments, each of the encoder and the decoder in the fiber tract segmentation model may be composed of various neural network modules, wherein the encoder can uses an advanced point cloud processing module. different from the fiber classification model in pre-processing, the advanced point cloud processing module directly reduces the dimensions of all points on a fiber into a global feature. In this application, a more advanced point cloud convolution operation is adopted. The point cloud convolution operation takes into account the spatial correlation of points on a fiber, thereby expressing the features of a fiber as a combination of the global feature and the feature of each point. In addition, for individual-level anatomical feature descriptors and the cluster-level anatomical feature descriptors, embedding layers of different dimensions are used to map the high-dimensional sparse matrix to a low-dimensional continuous vector, and the feature vector is spliced with the features obtained by point cloud convolution. In the decoder, a multi-layer perceptron is used for classification, and the features obtained by the encoder are fed to the multi-layer perceptron. Finally, through a probability regression layer, the probability of the category to which each fiber belongs is output. At last, the maximum probability is selected to obtain the category to which each fiber belongs, achieving the purpose of fine-grained and accurate segmentation of fiber tracts.

In order to obtain a trained fiber tract segmentation model, the method for automated brain white matter fiber tract segmentation may further include the following steps: building an initial fiber tract segmentation model; building a loss function and a sample data set for the model; and pre-training the initial fiber tract segmentation model based on the loss function and the sample data set of the model to obtain a trained fiber tract segmentation model.

Further, the loss function can be expressed as:

$$Loss = \frac{1}{N} {\sum }_{i} {Loss}_{i} = - \frac{1}{N} {\sum }_{i} {\sum }_{c = 1}^{M} {y}_{ic} log ( {p}_{i c} ) ;$$

where, M represents the number of categories into which the fiber tracts are segmented; M is 198 for the superficial white matter fibers and 602 for the deep white matter fibers; y_{ic} is 0 or 1, y_{ic} is 1 when a predicted category result of fiber i is the same as a real category result, and y_{ic} is 0 when the predicted category result of fiber i is different from the real category result; N represents a total number of fibers, and pic represents a probability that fiber i belongs to fiber tract category c and is obtained from the output of the decoder of the fiber tract segmentation model.

As an example, during the model training process, 100 pieces of fiber and anatomical feature descriptor data of a subject are acquired, and a white matter fiber tract segmentation data set is obtained and divided into 5 portions in equal proportion. Network training is performed by a five-fold cross-verification method to obtain a segmentation model with strong generalization and good robustness. Specifically, a learning rate is set to 0.0001, the training batch size is set to 2048, an Adam optimizer is used without setting weight decay, and the number of training epochs of the network is 500.

For the method for automated brain white matter fiber tract segmentation combined with anatomical priors disclosed in the above embodiments, the method may be evaluated by calculating the accuracy and F1 scores of fiber tracts as follows:

$$Accuracy = \frac{T P + T N}{T P + T N + F P + F N} ;$$

$$Precison = \frac{T P}{T P + F P} ;$$

$$Recall = \frac{T P}{T P + F N} ;$$

$$F 1 - score = \frac{2 \times Precision \times Recall}{P r e c i s i o n + R e c a l l} ;$$

where, TP is the number of positive examples correctly predicted by the model, TN is the number of negative examples correctly predicted by the model, FP is the number of negative examples incorrectly predicted by the model, and FN is the number of positive examples incorrectly predicted by the model.

Compared with other conventional methods, the fiber tract segmentation method of the present disclosure can improve the accuracy and F1 score by 2%-7%. In order to further test the effectiveness of the anatomical feature descriptors used in the method of the present disclosure, ablation experiments were conducted, and the results showed that both the individual-level anatomical feature descriptors and the cluster-level anatomical feature descriptors described in the method were effective, and the model with individual-level and cluster-level anatomical feature descriptions improved the accuracy by approximately 2%.

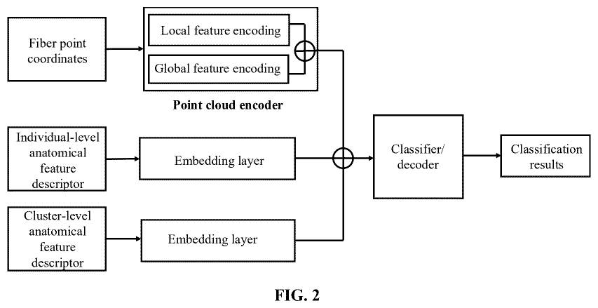

FIG. 2 is a schematic flow chart of the method for automated brain white matter fiber tract segmentation combined with anatomical priors according to another embodiment of the present disclosure. As shown in FIG. 2, the system corresponding to the method may specifically include a data pre-processing module, an individual-level anatomical feature descriptor acquisition module, a cluster-level anatomical feature descriptor acquisition module, a fiber tract segmentation model module, and a parameter and result export module.

In the data pre-processing module, the fiber data is classified into superficial white matter fibers and deep white matter fibers based on a pre-trained fiber classification neural network model, and outlier filtering is performed on the classified superficial white matter fibers, while the sample-averaged structural T1-weighted magnetic resonance images are processed to obtain an anatomical brain region division map. In the individual-level anatomical feature descriptor acquisition module, an affine transformation method is used to map each fiber onto the corresponding anatomical brain region division map, and the number of fiber points in each anatomical brain region is calculated to obtain individual-level anatomical feature descriptors. In the cluster-level anatomical feature descriptor acquisition module, a hierarchical clustering algorithm is used to cluster the individual-level anatomical feature descriptors, so that each fiber is clustered into a type of cluster, thus obtaining cluster-level anatomical feature descriptors. In the fiber tract segmentation model module, based on a point cloud neural network model, a neural network is designed to input geometric information on fiber point coordinates and the individual-level and cluster-level anatomical feature descriptors to obtain specific fiber classification results. For the parameter and result export module, a pre-training segmentation model may be exported for fiber tract segmentation on different test data. In addition, correctness rates, F1 scores, cross-entropy loss function and the like during the training process may also be output at the same time.

Through the above embodiments, it can be found that the method for automated brain white matter fiber tract segmentation combined with anatomical priors according to the present disclosure includes three parts: image pre-processing, anatomical feature descriptor calculation and segmentation model training. These three parts are performed in turn to form a complete fiber tract segmentation scheme. It is difficult to build feature descriptors from the anatomical brain region division map and fiber data of a single sample. The present disclosure adopts affine transformation to obtain the distribution of sampling points on a single fiber in the brain regions, also known as the individual-level distribution. Then, hierarchical clustering is performed to preliminarily classify the fibers on the individual-level distribution to obtain cluster-level anatomical features. That is, the individual-level and cluster-level distributions constitute an anatomical feature descriptor. In addition, this application also regards geometric information of fibers as point cloud, relies on an advanced artificial intelligence algorithm (point cloud neural network) to accelerate segmentation, and embeds the obtained anatomical feature descriptors. In this way, it solves the problems of large computing resource occupation and slow computing speed caused by the huge amount of data in conventional methods. In addition, by combining the anatomical brain region division map based on the structural T1-weighted magnetic resonance images with the whole-brain fiber data obtained by diffusion magnetic resonance imaging, this application provides a distribution description of anatomical brain regions of a single fiber and cluster-level fibers, thereby accurately and effectively representing the anatomical features of the fibers. In addition, this application embeds the anatomical distribution of each fiber and the anatomical distribution of cluster-level fibers into a neural network based on the geometric point cloud of fibers respectively, and trains the network to obtain a white matter fiber tract segmentation network model, thereby greatly improving the accuracy of automated fiber tract segmentation. That is, the method for automated brain white matter fiber tract segmentation disclosed in this application can accurately segment fiber tracts connecting different brain regions, and further segment large fiber tracts into small clusters, thereby providing a foundation for subsequent brain research and medical diagnosis and achieving broad biomedical application prospects. The method for automated brain white matter fiber tract segmentation disclosed in this application can segment whole-brain fibers in different data sets without modifying the underlying training parameters, and is universal for whole-brain fiber data of patients with Alzheimer's disease, mild cognitive impairment and the like.

Correspondingly, further disclosed is a system for automated brain white matter fiber tract segmentation combined with anatomical priors, including: a processor, a memory and a computer program stored on the memory. The processor is configured to execute the computer program, and the system implements the steps of the method as described in any of the above embodiments when the computer program is executed.

Embodiments of the present disclosure also provide a computer-readable storage medium having stored thereon a computer program. When the computer program is executed by a processor, the steps of the method described above are implemented. The computer-readable storage medium may be a tangible storage medium such as a random access memory (RAM), a memory, a read-only memory (ROM), an electrically programmable ROM, an electrically erasable programmable ROM, a register, a floppy disk, a hard disk, a removable storage disk, a CD-ROM, or any other form of storage medium known in the art.

Those of ordinary skill in the art will appreciate that each exemplary component, system, and method described in connection with the embodiments disclosed herein can be implemented in the form of hardware, software, or a combination of both. Whether it is implemented in the form of hardware or software depends on the specific application and design constraints of the technical solution. Those skilled in the art may use different methods to achieve the described functions for each particular application, but such implementation should not be considered beyond the scope of the present disclosure. When implemented in the form of hardware, it may be, for example, an electronic circuit, an application specific integrated circuit (ASIC), appropriate firmware, plug-in, function card, etc. When implemented in the form of software, the elements of the present disclosure are programs or code segments that are used to perform the required tasks. The program or code segments may be stored in a machine-readable medium or transmitted over a transmission medium or communication link via a data signal carried in a carrier wave.

It should be clear that the present disclosure is not limited to the specific configuration and processing described above and shown in the figures. For the sake of simplicity, a detailed description of the known method is omitted here. In the above embodiments, several specific steps are described and shown as examples. However, the method process of the present disclosure is not limited to the specific steps described and shown. Those skilled in the art can make various changes, modifications and additions, or change the order of the steps after understanding the spirit of the present disclosure.

In the present disclosure, the features described and/or illustrated for one embodiment may be used in the same manner or in a similar manner in one or more other embodiments, and/or combined with or replace the features of other embodiments.

The above are only preferred embodiments of the present disclosure and are not intended to limit the present disclosure. For those skilled in the art, the embodiments of the present disclosure may have various modifications and changes. Any modification, equivalent replacement, improvement, etc. made within the spirit and principle of the present disclosure shall fall within the scope of the present disclosure.

What is claimed is:

1. A method for automated brain white matter fiber tract segmentation combined with anatomical priors, comprising the following steps:
obtaining whole-brain fiber point coordinates and structural T1-weighted magnetic resonance images, determining superficial white matter fibers and deep white matter fibers based on the whole-brain fiber point coordinates, and generating an anatomical brain region division map based on the structural T1-weighted magnetic resonance images;
determining an individual-level anatomical feature descriptor of each fiber based on the superficial white matter fibers, the deep white matter fibers and the anatomical brain region division map, and respectively determining a cluster-level anatomical feature descriptor corresponding to each fiber based on the individual-level anatomical feature descriptor of each of the superficial white matter fibers and the individual-level anatomical feature descriptor of each of the deep white matter fibers; and
inputting the whole-brain fiber point coordinates, the individual-level anatomical feature descriptors and the cluster-level anatomical feature descriptors into a trained fiber tract segmentation model, and obtaining classification results of fiber tracts based on the trained fiber tract segmentation model;
wherein determining an individual-level anatomical feature descriptor of each fiber based on the superficial white matter fibers, the deep white matter fibers and the anatomical brain region division map comprises:
performing 3D affine transformation on each of the superficial white matter fibers and the deep white matter fibers, and mapping fiber points of each fiber to divided brain regions of the anatomical brain region division map to obtain an anatomical distribution of the fiber points of each fiber; and
obtaining an individual-level anatomical feature descriptor of each fiber based on the anatomical distribution of fiber points of each fiber; and
wherein respectively determining a cluster-level anatomical feature descriptor corresponding to each fiber based on the individual-level anatomical feature descriptor of each of the superficial white matter fibers and the individual-level anatomical feature descriptor of each of the deep white matter fibers comprises:
performing hierarchical clustering on the superficial white matter fibers and the deep white matter fibers respectively based on the individual-level anatomical feature descriptor of each of the superficial white matter fibers and the individual-level anatomical feature descriptor of each of the deep white matter fibers;
determining the number of clusters corresponding to the superficial white matter fibers and the number of clusters corresponding to the deep white matter fibers; and
determining the clusters to which various fibers belong based on the number of clusters corresponding to the superficial white matter fibers, the number of clusters corresponding to the deep white matter fibers and hierarchical clustering results, and determining a cluster-level anatomical feature descriptor corresponding to each fiber based on the cluster to which each fiber belongs.

2. The method for automated brain white matter fiber tract segmentation combined with anatomical priors according to claim 1, wherein the fiber tract segmentation model comprises a point cloud encoder module, a first embedding layer, a second embedding layer and a decoder module.

3. The method for automated brain white matter fiber tract segmentation combined with anatomical priors according to claim 2, wherein the anatomical brain region division map contains 286 brain regions.

4. The method for automated brain white matter fiber tract segmentation combined with anatomical priors according to claim 2, wherein inputting the whole-brain fiber point coordinates, the individual-level anatomical feature descriptors and the cluster-level anatomical feature descriptors into a trained fiber tract segmentation model, and obtaining classification results of fiber tracts based on the trained fiber tract segmentation model comprises:
performing local feature encoding and global feature encoding on the whole-brain fiber point coordinates on the basis of the point cloud encoder, and fusing the local feature encoding and global feature encoding to obtain feature codes;
inputting the individual-level anatomical feature descriptors into the first embedding layer to obtain first embedding codes;
inputting the cluster-level anatomical feature descriptors into the second embedding layer to obtain second embedding codes;
fusing the feature codes, the first embedding codes and the second embedding codes to obtain fused codes; and
inputting the fused codes to the decoder to obtain classification results of fiber tracts.

5. The method for automated brain white matter fiber tract segmentation combined with anatomical priors according to claim 2, further comprises:
building an initial fiber tract segmentation model;
building a loss function and a sample data set for the model; and
pre-training the initial fiber tract segmentation model based on the loss function and the sample data set of the model to obtain a trained fiber tract segmentation model.

6. The method for automated brain white matter fiber tract segmentation combined with anatomical priors according to claim 5, wherein the loss function is:
$$Loss = \frac{1}{N} {\sum }_{i} {Loss}_{i} = - \frac{1}{N} {\sum }_{i} {\sum }_{c = 1}^{M} {y}_{ic} l og ( {p}_{i c} ) ;$$
where, M represents the number of categories into which the fiber tracts are segmented; y_{ic} is 0 or 1, y_{ic} is 1 when a predicted category result of fiber i is the same as a real category result, and y_{ic} is 0 when the predicted category result of fiber i is different from the real category result; N represents a total number of fibers, and pic represents a probability that fiber i belongs to fiber tract category c.

7. The method for automated brain white matter fiber tract segmentation combined with anatomical priors according to claim 1, wherein determining superficial white matter fibers and deep white matter fibers based on the whole-brain fiber point coordinates comprises:
inputting the whole-brain fiber point coordinates into a trained fiber classification model for pre-classification to obtain a superficial white matter fiber pre-classification result and a deep white matter fiber pre-classification result;
inputting superficial white matter fibers in the superficial white matter fiber pre-classification result into a fiber filtration model to obtain false superficial white matter fibers; and
labeling the false superficial white matter fibers as deep white matter fibers.

8. A system for automated brain white matter fiber tract segmentation combined with anatomical priors, comprising: a processor, a memory and a computer program stored on the memory, wherein the processor is configured to execute the computer program, and the system implements the steps of:
obtaining whole-brain fiber point coordinates and structural T1-weighted magnetic resonance images, determining superficial white matter fibers and deep white matter fibers based on the whole-brain fiber point coordinates, and generating an anatomical brain region division map based on the structural T1-weighted magnetic resonance images;
determining an individual-level anatomical feature descriptor of each fiber based on the superficial white matter fibers, the deep white matter fibers and the anatomical brain region division map, and respectively determining a cluster-level anatomical feature descriptor corresponding to each fiber based on the individual-level anatomical feature descriptor of each of the superficial white matter fibers and the individual-level anatomical feature descriptor of each of the deep white matter fibers; and
inputting the whole-brain fiber point coordinates, the individual-level anatomical feature descriptors and the cluster-level anatomical feature descriptors into a trained fiber tract segmentation model, and obtaining classification results of fiber tracts based on the trained fiber tract segmentation model;
wherein determining an individual-level anatomical feature descriptor of each fiber based on the superficial white matter fibers, the deep white matter fibers and the anatomical brain region division map comprises:
performing 3D affine transformation on each of the superficial white matter fibers and the deep white matter fibers, and mapping fiber points of each fiber to divided brain regions of the anatomical brain region division map to obtain an anatomical distribution of the fiber points of each fiber; and
obtaining an individual-level anatomical feature descriptor of each fiber based on the anatomical distribution of fiber points of each fiber; and
wherein respectively determining a cluster-level anatomical feature descriptor corresponding to each fiber based on the individual-level anatomical feature descriptor of each of the superficial white matter fibers and the individual-level anatomical feature descriptor of each of the deep white matter fibers comprises:
performing hierarchical clustering on the superficial white matter fibers and the deep white matter fibers respectively based on the individual-level anatomical feature descriptor of each of the superficial white matter fibers and the individual-level anatomical feature descriptor of each of the deep white matter fibers;
determining the number of clusters corresponding to the superficial white matter fibers and the number of clusters corresponding to the deep white matter fibers; and
determining the clusters to which various fibers belong based on the number of clusters corresponding to the superficial white matter fibers, the number of clusters corresponding to the deep white matter fibers and hierarchical clustering results, and determining a cluster-level anatomical feature descriptor corresponding to each fiber based on the cluster to which each fiber belongs.

9. The system for automated brain white matter fiber tract segmentation combined with anatomical priors according to claim 8, wherein the fiber tract segmentation model comprises a point cloud encoder module, a first embedding layer, a second embedding layer and a decoder module.

10. The system for automated brain white matter fiber tract segmentation combined with anatomical priors according to claim 9, wherein the anatomical brain region division map contains 286 brain regions.

11. The system for automated brain white matter fiber tract segmentation combined with anatomical priors according to claim 9, wherein inputting the whole-brain fiber point coordinates, the individual-level anatomical feature descriptors and the cluster-level anatomical feature descriptors into a trained fiber tract segmentation model, and obtaining classification results of fiber tracts based on the trained fiber tract segmentation model comprises:
performing local feature encoding and global feature encoding on the whole-brain fiber point coordinates on the basis of the point cloud encoder, and fusing the local feature encoding and global feature encoding to obtain feature codes;
inputting the individual-level anatomical feature descriptors into the first embedding layer to obtain first embedding codes;
inputting the cluster-level anatomical feature descriptors into the second embedding layer to obtain second embedding codes;
fusing the feature codes, the first embedding codes and the second embedding codes to obtain fused codes; and
inputting the fused codes to the decoder to obtain classification results of fiber tracts.

12. The system for automated brain white matter fiber tract segmentation combined with anatomical priors according to claim 8, further comprises:
building an initial fiber tract segmentation model;
building a loss function and a sample data set for the model; and
pre-training the initial fiber tract segmentation model based on the loss function and the sample data set of the model to obtain a trained fiber tract segmentation model.

13. The system for automated brain white matter fiber tract segmentation combined with anatomical priors according to claim 1, wherein determining superficial white matter fibers and deep white matter fibers based on the whole-brain fiber point coordinates comprises:
inputting the whole-brain fiber point coordinates into a trained fiber classification model for pre-classification to obtain a superficial white matter fiber pre-classification result and a deep white matter fiber pre-classification result;
inputting superficial white matter fibers in the superficial white matter fiber pre-classification result into a fiber filtration model to obtain false superficial white matter fibers; and
labeling the false superficial white matter fibers as deep white matter fibers.

14. A computer-readable storage medium having stored thereon a computer program, when the computer program is executed by a processor, the following steps are implemented:
obtaining whole-brain fiber point coordinates and structural T1-weighted magnetic resonance images, determining superficial white matter fibers and deep white matter fibers based on the whole-brain fiber point coordinates, and generating an anatomical brain region division map based on the structural T1-weighted magnetic resonance images;
determining an individual-level anatomical feature descriptor of each fiber based on the superficial white matter fibers, the deep white matter fibers and the anatomical brain region division map, and respectively determining a cluster-level anatomical feature descriptor corresponding to each fiber based on the individual-level anatomical feature descriptor of each of the superficial white matter fibers and the individual-level anatomical feature descriptor of each of the deep white matter fibers; and
inputting the whole-brain fiber point coordinates, the individual-level anatomical feature descriptors and the cluster-level anatomical feature descriptors into a trained fiber tract segmentation model, and obtaining classification results of fiber tracts based on the trained fiber tract segmentation model;
wherein determining an individual-level anatomical feature descriptor of each fiber based on the superficial white matter fibers, the deep white matter fibers and the anatomical brain region division map comprises:
performing 3D affine transformation on each of the superficial white matter fibers and the deep white matter fibers, and mapping fiber points of each fiber to divided brain regions of the anatomical brain region division map to obtain an anatomical distribution of the fiber points of each fiber; and
obtaining an individual-level anatomical feature descriptor of each fiber based on the anatomical distribution of fiber points of each fiber; and
wherein respectively determining a cluster-level anatomical feature descriptor corresponding to each fiber based on the individual-level anatomical feature descriptor of each of the superficial white matter fibers and the individual-level anatomical feature descriptor of each of the deep white matter fibers comprises:
performing hierarchical clustering on the superficial white matter fibers and the deep white matter fibers respectively based on the individual-level anatomical feature descriptor of each of the superficial white matter fibers and the individual-level anatomical feature descriptor of each of the deep white matter fibers;
determining the number of clusters corresponding to the superficial white matter fibers and the number of clusters corresponding to the deep white matter fibers; and
determining the clusters to which various fibers belong based on the number of clusters corresponding to the superficial white matter fibers, the number of clusters corresponding to the deep white matter fibers and hierarchical clustering results, and determining a cluster-level anatomical feature descriptor corresponding to each fiber based on the cluster to which each fiber belongs.
